(19) United States
(12) Patent Application Publication
(10) Pub. No.: US 2009/0110593 A1
(43) Pub. Date: Apr. 30, 2009

(54) Foamed Materials Based on Aminoplasts as Sterilizable Raw Materials

(75) Inventor: Hans-Jurgen Quadbeck-Seeger, Bad Durkheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/226,410

(22) PCT Filed: Apr. 5, 2007
(86) PCT No.: PCT/EP2007/053358

§ 371 Date: Oct. 17, 2008

(30) Foreign Application Priority Data
Apr. 18, 2006 (EP) 06112698.3

(51) Int. Cl. A61L 2/12; A61L 2/04; A61L 2/07

(52) U.S. Cl. 422/21; 422/1; 422/26

(57) ABSTRACT

Use of an open-celled foam based on an aminoplastic as sterilizable working material, and also methods of sterilizing the open-celled foam by impregnation with a microbicidal liquid such as alcohol or formalin, methods of sterilizing working vessels for medical or microbiological work and for decontaminating material contaminated with micro-organisms at temperatures above 100° C.

The invention relates to the use of an open-celled foam based on an aminoplastic as sterilizable working material, and also methods of sterilizing the open-celled foam by impregnation with a microbicidal liquid such as alcohol or formalin, methods of sterilizing working vessels for medical or microbiological work and for decontaminating material contaminated with microorganisms at temperatures above 100° C.

In microbiology, working under sterile conditions and the sterilization of working material play an important role. For such work, materials such as absorbent cotton plugs for closing bottles and underlays are sterilized by heating at 128° C. and a pressure of 4 atmospheres in pressure-rated sterilizers and marketed in packed form.

The disposal of contaminated material is likewise of importance. Particularly when working with pathogenic microorganisms and genetically engineered microorganisms, strict regulations apply.

Bacteria and fungi are cultured on solid or in liquid nutrient media. These have to be sterilized before inoculation. To kill microorganisms, they are generally treated at high temperatures in an autoclave. Sterile or sterilizable containers are usually protected against infection by means of absorbent cotton stoppers or woodpulp stoppers, overlapping lids or screw closures. Glass and metal equipment can be sterilized dry in metal containers or other heat-resistant containers. Glass pipettes are sterilized dry in special cans.

Culture tubes, bottles and flasks which are to be kept sterile are closed with stoppers of absorbent cotton, rolled woodpulp or silicone foam which allow access of air but act as a deep bed filter to keep out airborne microorganisms. However, they must comprise no moisture because otherwise microorganisms can grow from the outside through to the inside. Over relatively short times of about 2-3 days, sterility is, as in the case of Petri dishes, also ensured by tight-fitting overlapping caps of, for example, aluminum.

It was an object of the invention to provide a working material for microbiology which can be sterilized in a simple fashion. The working material should, in particular, be suitable as closure for microbiological working vessels and be able to be sterilized at high temperatures even at atmospheric pressure.

We have accordingly found the use of an open-celled foam based on an aminoplastic as sterilizable working material.

As open-celled foams, preference is given to using elastic foams based on a melamine-formaldehyde condensation product having a specific density of from 5 to 100 g/l, in particular from 8 to 20 g/l. The cell count is usually in the range from 50 to 300 cells/25 mm. The tensile strength is preferably in the range from 100 to 150 kPa and the elongation at break is preferably in the range from 8 to 20%.

To produce it, a highly concentrated solution or dispersion of a melamine-formaldehyde precondensate comprising blowing agent can be foamed and cured by means of hot air, steam or microwave radiation as described in EP-A 071 672 or EP-A 037 470. Such foams are commercially available under the name Basotect® from BASF Aktiengesellschaft.

The molar ratio of melamine to formaldehyde is generally in the range from 1:1 to 1:5. To produce particularly low-formaldehyde foams, the molar ratio is selected in the range from 1:1.3 to 1:1.8 and a precondensate which is free of sulfite groups is used, as described, for example, in WO 01/94436.

To improve the use properties, the foams can subsequently be heat treated and pressed. The foams can be cut to the desired shape and thickness and laminated with covering layers on one or both sides. For example, a polymer film or metal foil can be applied as covering layer.

The open-celled foam can, owing to the substantial chemical resistance of the melamine-formaldehyde condensate, also come into direct contact with various chemicals or cryogenic liquids.

The foam can have been made hydrophobic. The addition of hydrophobicizing agents can be effected during the foam formation or by after-impregnation with a hydrophobicizing agent. Suitable hydrophobicizing agents are, for example, silicones, paraffins, fluorocarbon resins or fluorinated or silicone surfactants. Since microbiological work is mostly carried out in aqueous media, hydrophobicizing treatment of the foam, as described in EP-A 633 283, is in many cases advantageous for reducing water absorption.

The foam can be combined with yarns and woven fabrics produced therefrom comprising from 5 to 90% by weight of melamine fibers, from 5 to 90% by weight of natural fibers and from 0.1 to 30% by weight of polyamide fibers, as are described in WO 02/10492. Owing to the high thermal, chemical and mechanical stability of these fibers, it is possible, for example, to increase the strength of the foam without having an adverse effect on the sterilizability.

For this reason, the sterilization of the open-celled foam based on an aminoplastic for microbiological work can be carried out simply by impregnation with a microbicidal liquid such as alcohol or formalin. In contrast to an absorbent cotton plug or woodpulp plug, the open-celled foam takes up the microbicidal liquid very quickly and can, after an appropriate contact time, be emptied again by application of pressure or reduced pressure. The sterilized open-celled foam can subsequently be, for example, reused as closure or be disposed of.

Owing to the elasticity of the open-celled foam, it can be inserted into prefabricated container parts in a simple manner. Even at low temperatures, for example below −80° C., the foam remains elastic. Damage as a result of embrittlement does not occur.

According to the invention, the opening of a working vessel, for example a culture tube, bottle or flask for medical or microbiological work, can be closed by means of the open-celled foam based on an aminoplastic and be treated at temperatures above 100° C. for the purposes of sterilization.

It is also possible to introduce material contaminated with microorganisms into a working vessel, close the vessel by means of the open-celled foam based on an aminoplastic and decontaminate the vessel by treatment at temperatures above 100° C.

The treatment can be carried out under steam at from 120 to 140° C. and a pressure in the range from 1 to 4 bar in an autoclave. The air has to be displaced completely from the interior of the autoclave by means of steam. To achieve successful sterilization, it is the temperature level which is important, not the gauge pressure which is necessary to achieve temperatures above 100° C. 134° C. can only be employed in the case of suitably insensitive materials.

To sterilize small volumes of liquid of less than 20 ml, it is generally necessary to heat at 121° C. for at least 15 minutes pure sterilization time without the heating time required for the material to be sterilized. In the case of individual volumes of from about 50 ml to 1 l, heating times of from 5 to 40 minutes have to be added. In modern autoclaves, the sterilization time set is controlled by means of a temperature sensor in the material being sterilized.

The vessels to be sterilized must not be closed tightly so that air and water vapor escape on heating and can flow in on cooling. Otherwise empty vessels should comprise some water so that they fill with steam. The open-celled foam used according to the invention is found to be advantageous here, since it does not have to be protected from dripping condensate water by means of parchment paper or aluminum foil as in the case of absorbent cotton stoppers and woodpulp stoppers. All closures, including screw closures, must also not become moist from the inside, which can occur, for example, as a result of liquid boiling up. For this reason, vessels may only be filled to an extent of not more than ¾. After the sterilization time has elapsed, the autoclave has to be opened and cooled to below 100° C. so that liquids under pressure do not begin to boil. Autoclaves nowadays have to be secured against opening at temperatures above 80° C.

However, the treatment is preferably carried out dry, i.e. without steam, at a temperature in the range from 140 to 220° C. Expense and working steps can be reduced considerably as a result. Glass and metal equipment can, when packed in parchment paper or aluminum foil, be sterilized dry. 150° C. for 3 hours or 180° C. for 30 minutes is usually sufficient for this purpose. The heating times to be added on depend on the size of the individual parts and on the degree of fill of the interior of the sterilizer. In the case of large masses to be heated up, these are a number of hours. It is most advantageous to commence the sterilization in the afternoon so that the sterilizer can cool slowly overnight. Glass pipettes are sterilized in special two-piece aluminum or stainless steel cans.

Sterilizers are distinguished from pure drying ovens by the presence of a fan to circulate the air, which should be switched on especially when the material to be sterilized is tightly packed.

The foam used according to the invention has a high thermal stability but does not absorb microwave radiation. It is therefore also suitable for sterilization by introduction of microwave energy.

After sterile vessels or vessels comprising pure cultures are opened and before they are closed again, the opening and stopper are briefly flamed with a bunsen burner to sterilize them. In contrast to the open-celled foam used according to the invention, loosely stuffed absorbent cotton stoppers can catch fire during this procedure.

The open-celled foam used according to the invention has a high thermal stability at temperatures up to 180° C. It is easy to sterilize in conventional sterilizers. It can even be heated briefly to 220° C. Since microorganisms generally do not survive heating to 150° C., the open-celled foam used according to the invention can also be heated for the necessary time at from 150° to 200° C. under atmospheric pressure in a laboratory oven or a baking oven, which are available as table-mounted models. Expense and working steps can be reduced considerably as a result. The foam is highly compressible and elastic, and it is therefore possible to fit even pieces which have been roughly cut to size into openings of variable diameter.

### EXAMPLES

The following materials were used as stoppers in the culture of microorganisms:

### Example 1

Stoppers which were composed of an open-celled melamine-formaldehyde foam having a density of about 10 kg/m³ (Basotect® from BASF Aktiengesellschaft) and had been cut to size.

### Comparative Experiment C1

Conventional absorbent cotton stoppers (27-32.5 mm; Buddeberg, # 1012700)

### Comparative Experiment C2

Plastic stoppers having a permeable membrane (Buddeberg)

### 1. Ability to be Flamed

Absorbent cotton stoppers (C1) are completely unsuitable for sterilization by means of a flame and begin to burn immediately. In contrast, plastic stoppers (C2) and Basotect® stoppers (example 1) can be pretreated appropriately if required.

### 2. Autoclaveability/Sterility

The stoppers according to the invention (example 1) and the comparative stoppers (C1, C2) were autoclaved under standard conditions (121° C./20 min; dry or moist heat) and incubated in conical flasks filled with LB growth medium without addition of microorganisms at 37° C., 150 rpm for 7 days. In none of the batches was an alteration of the stopper as a result of the heat treatment or contamination in the medium during incubation observed.

### 3. Oxygen Permeability

The oxygen permeability of the 3 different stoppers was detected by means of a sulfite detection system. For this purpose, 100 ml of sulfite solution were introduced into a 1 l conical flask and incubated at RT/150 rpm. The time at which the color changes is a measure of the oxygen permeability.

### Preparation of the 0.5 M Sulfite System

The standard 0.5 M sulfite system (sodium sulfite ≧98%, catalogue No. 60860, Roth, Karlsruhe) is made up with 0.012 M of phosphate buffer; 10⁻⁷ M cobalt sulfate; 0.015 g/l of bromothymol blue solution (catalogue No. 18460, Fluka, Buchs/CH) with nitrogen-degassed (5-10 min) deionized water using the method of Hermann et al (Biotech Bioeng, 2001).

To prepare 1000 ml of 0.5 M sulfite solution, 24 ml of 0.5 M phosphate buffer solution (see below) are made up to 1000 ml. 800 ml of this 0.012 M phosphate buffer solution are sparged with nitrogen. 63 g of sodium sulfite are dissolved in the 800 ml of solution and 15 ml of bromothymol blue solution having a concentration of 1 g/l are added (see below). 2 ml of 5.10⁻⁵ M cobalt solution are added and the mixture is made up to 1000 ml with the remainder of the 0.012 M phosphate buffer solution. The pH is set to 8 by means of sulfuric acid (30%, catalogue No. A2712.500, Applichem, Darmstadt). During the preparation, the solution is continually sparged with nitrogen.

### i) Preparation of the 0.5 M Phosphate Buffer Solution

### Method for 500 ml of 0.5 M Na₂HPO₄ Solution

35.490 g of Na₂HPO₄ (disodium hydrogenphosphate, anhydrous ≧99%, catalogue No. P030.1, Roth, Karlsruhe) are dissolved in 450 ml of deionized water and made up to 500 ml.

### Method for 50 ml of 0.5 M NaH₂PO₄ Solution

3.450 g of NaH₂PO₄ (sodium dihydrogenphosphate, monohydrate ≧98%, catalogue No. K300.2, Roth, Karlsruhe) are dissolved in 45 ml of deionized water and made up to 50 ml.

The 500 ml of 0.5 M Na₂HPO₄ solution made up are brought to pH=8 by addition of the 0.5 M NaH₂PO₄ solution (about 40-50 ml).

### ii) Preparation of the Cobalt Catalyst

5.10⁻³ mol/l (stock solution): 140.55 mg of CoSO₄*7H₂O (≧97.5%, catalogue No. 60860, Fluka Chemie AG, Buchs/CH) are made up to 100 ml with deionized water.

5.10⁻³ mol/l (final solution): 1 ml of the above stock solution are made up to 100 ml with deionized water.

iii) Preparation of the Bromothymol Blue Solution

0.1 g of bromothymol blue (catalogue No. 18460, Fluka, Buchs/CH) is made up to 100 ml with water and stirred for about 1 h (=1 g/l).

Plastic stoppers (C2) and Basotect® stoppers (example 1) display similar behavior (color change after 11-13 hours), while absorbent cotton stoppers (C1) are significantly poorer at changing color after 17-20 hours. Limitation of the oxygen transfer can lead to unsatisfactory growth or changes in the metabolism of the microorganisms and is undesirable. Basotect® or plastic stoppers are thus significantly better.

### 4. Evaporation Losses

A number of batches were incubated in 1 l conical flasks, in each case filled with 150 ml of LB medium, at 37° C./150 rpm. The evaporation losses after 7 days were about 10-11 ml in the case of the absorbent cotton stoppers (C1) and plastic stoppers (C2), while about 12-14 ml are lost through the coarse-pored Basotect®. The minimally increased evaporation is more than made up for by the other positive properties of Basotect® and is not of a critical magnitude.

### 5. Handling

Absorbent cotton stoppers (C1) and also plastic stoppers (C2) are very rigid and there is a risk of them falling off from the flasks again (especially during shaking at a high frequency). When these stoppers are utilized with application of a high force, there is also a potential danger of injury. Basotect® stoppers can easily be inserted into the openings of the flasks with application of minimal force and are optimally matched.

1. A method of sterilizing working vessels by treatment at temperatures above 100° C., wherein the opening of the working vessel is closed by means of an open-celled foam based on an aminoplastic.

2. A method of decontaminating material contaminated with microorganisms by treatment at temperatures above 100° C., wherein the material is introduced into a working vessel before the treatment and the vessel is closed by means of an open-celled foam based on an aminoplastic.

3. The method according to claim 1, wherein the opening of the working vessel is closed by means of an open-celled foam based on a melamine-formaldehyde condensation product.

4. The method according to claim 1, wherein the open-celled foam has a specific density in the range from 5 to 100 g/l.

5. The method according to claim 1, wherein the open-celled foam has been produced from a melamine-formaldehyde condensation product having a molar ratio of melamine to formaldehyde in the range from 1:1 to 1:5.

6. The method according to claim 1, wherein the open-celled foam has been treated with a hydrophobicizing agent.

7. The method according to claim 1, wherein a culture tube, bottle or flask for medical or microbiological work is used as working vessel.

8. The method according to claim 1, wherein the treatment is carried out under steam at from 120 to 140° C. and a pressure in the range from 1 to 4 bar in an autoclave.

9. The method according to claim 1, wherein the treatment is carried out dry at a temperature in the range from 140 to 220° C.

10. The method according to claim 1, wherein the treatment is carried out using microwave energy.

11. The method according to claim 2, wherein the opening of the working vessel is closed by means of an open-celled foam based on a melamine-formaldehyde condensation product.

12. The method according to claim 2, wherein the open-celled foam has a specific density in the range from 5 to 100 g/l.

13. The method according to claim 3, wherein the open-celled foam has a specific density in the range from 5 to 100 g/l.

14. The method according to claim 2, wherein the open-celled foam has been produced from a melamine-formaldehyde condensation product having a molar ratio of melamine to formaldehyde in the range from 1:1 to 1:5.

15. The method according to claim 3, wherein the open-celled foam has been produced from a melamine-formaldehyde condensation product having a molar ratio of melamine to formaldehyde in the range from 1:1 to 1:5.

16. The method according to claim 4, wherein the open-celled foam has been produced from a melamine-formaldehyde condensation product having a molar ratio of melamine to formaldehyde in the range from 1:1 to 1:5.

17. The method according to claim 2, wherein the open-celled foam has been treated with a hydrophobicizing agent.

18. The method according to claim 3, wherein the open-celled foam has been treated with a hydrophobicizing agent.

19. The method according to claim 4, wherein the open-celled foam has been treated with a hydrophobicizing agent.

20. The method according to claim 5, wherein the open-celled foam has been treated with a hydrophobicizing agent.
